(19) United States
(12) Patent Application Publication
(10) Pub. No.: US 2021/0403637 A1
(43) Pub. Date: Dec. 30, 2021

(54) CURABLE RESIN COMPOSITION AND FIBER REINFORCED RESIN MATRIX COMPOSITE MATERIAL

(71) Applicant: Cytec Industries Inc., Princeton, NJ (US)

(72) Inventor: Jonathan E. MEEGAN, Chester (GB)

(21) Appl. No.: 17/293,072

(22) PCT Filed: Nov. 13, 2019
(86) PCT No.: PCT/US2019/061104

§ 371 Date: May 12, 2021

Related U.S. Application Data
(60) Provisional application No. 62/760,551, filed on Nov. 13, 2018.

(51) Int. Cl. C08G 59/50; C07C 213/10; C07C 209/90; C07C 315/06; C08J 5/00

(52) U.S. Cl. CPC C08G 59/5033; C07C 213/10; C08J 2363/00; C07C 315/06; C08J 5/005; C07C 209/90

(57) ABSTRACT

A curing agent composition contains at least one multifunctional aromatic amine that forms a crystalline solid at 25° C., and at least one halo-substituted diethyltoluenediamine, in an amount effective to inhibit crystallization of the at least one multifunctional aromatic amine. A curable resin composition contains at least one epoxy compound having at least two epoxide groups per molecule of the epoxy compound and the curing agent composition. Methods for inhibiting phase separation of a curing agent composition or curable resin composition that contains at least one multifunctional aromatic amine that forms a crystalline solid at 25° C. include a step of adding to the respective composition at least one halo-substituted diethyltoluenediamine in an amount effective to inhibit crystallization of the at least one multifunctional aromatic amine. The compositions and methods are useful in making fiber reinforced resin matrix composite articles.

### FIELD OF THE INVENTION

This invention relates to a curable resin, fiber reinforced resin matrix composite materials comprising fibers and the curable resin, and fiber reinforced resin matrix composite articles made thereby.

### BACKGROUND OF THE INVENTION

Carbon and/or glass reinforced carbon fiber epoxy resin matrix composite materials are used in high performance applications, such as in aerospace and automotive applications, where lightweight high performance are required.

Fiber reinforced epoxy resin matrix composite parts can be manufactured by different methods, including laying up and curing composite prepreg, filament winding, resin transfer molding and vacuum assisted resin transfer molding.

In some methods, particularly filament winding, resin transfer molding and vacuum assisted resin transfer molding, relatively low viscosity resin matrix material is used. The liquid resin composition may be in the form of a single component composition comprising a mixture of curable epoxy resin and one or more curing agents, or of a two component system in which a curable epoxy resin component is mixed with a separate curing agent composition immediately prior to use.

Single component liquid epoxy resin formulations offer consistency and ease of use in that there is no requirement for the user to mix separate resin formulation components prior to use of the resin formulation, but require a balance of shelf life stability and reactivity. Two component liquid epoxy resin formulations offer versatility in regard to balancing shelf life stability and reactivity, but require mixing immediately prior to use, but the quality and consistency of the final resin composition are sensitive to temperature and mixing conditions.

Single component liquid epoxy resin formulations containing curable epoxy resins and multifunctional aromatic amine hardeners have been found to offer good performance, except for a tendency to phase separate into epoxy-rich and crystalline hardener phases during storage at ambient temperature. The crystalline hardener phase must be dissolved and re-disbursed in the liquid resin formulation, for example, by mixing the resin formulation, with heating, prior to use. The need for such processing of such formulations immediately prior to use negates a significant part of the advantage of using a single component liquid epoxy resin formulation.

In two component epoxy resin formulations for which the curing agent component comprises a multifunctional aromatic amine, crystallization of the multifunctional aromatic amine can complicate handling of the curing agent component, e.g., by requiring melting and mixing of the curing agent prior to use.

There is an interest in producing one component liquid epoxy resin formulations and multifunctional aromatic amine compositions for use as the curing agent component of a two component epoxy resin formulation that, in each case, offer improved storage stability in order to minimize the need for pre-use processing of such liquid epoxy resin formulations, as well as good reactivity and good final resin properties.

### SUMMARY OF THE INVENTION

In a first aspect, the present invention is directed to a curing agent composition, comprising:

at least one multifunctional aromatic amine that is that forms a crystalline solid at 25° C., and
at least one halo-substituted diethyltoluenediamine, in an amount effective to inhibit crystallization of the at least one multifunctional aromatic amine.

In a second aspect, the present invention is directed to a method for inhibiting phase separation of a curing agent, comprising adding to a curing agent that comprises at least one multifunctional aromatic amine that is that forms a crystalline solid at 25° C., at least one halo-substituted diethyltoluenediamine in an amount effective to inhibit crystallization of the at least one multifunctional aromatic amine.

In a third embodiment, the present invention is directed to a process for making a curable resin composition, comprising mixing the curing agent composition of claim 1 and at least one epoxy compound having at least two epoxide groups per molecule of the epoxy compound.

In a fourth aspect, the present invention is directed to a curable resin composition, comprising:

at least one epoxy compound having at least two epoxide groups per molecule of the epoxy compound,
at least one multifunctional aromatic amine that is that forms a crystalline solid at 25° C., and
at least one halo-substituted diethyltoluenediamine, in an amount effective to inhibit crystallization of the at least one multifunctional aromatic amine.

In a fifth aspect, the present invention is directed to a method for inhibiting phase separation of a curable resin composition, comprising adding to a curable resin composition comprising at least one epoxy compound having at least two epoxide groups per molecule of the epoxy compound and at least one curing agent that comprises at least one multifunctional aromatic amine that forms a crystalline solid at 25° C., at least one halo-substituted diethyltoluenediamine in an amount effective to inhibit crystallization of the at least one multifunctional aromatic amine.

In a sixth aspect, the present invention is directed to a curable fiber reinforced resin matrix composite article, comprising fibers and a curable resin composition according to claim 4.

The crystallization inhibiting amount of the at least one halo-substituted diethyltoluenediamine inhibits crystallization of the at least one multifunctional aromatic amine component of the epoxy resin composition, which, compared to an analogous epoxy resin composition that lacks the at least one halo-substituted diethyltoluenediamine, reduces phase separation of such composition during storage and improves the storage stability of such composition.

### BRIEF DESCRIPTION OF THE DRAWINGS

FIG. 1 shows a table comparing the % recrystallization over time for compositions, each comprising a multifunctional aromatic amine that forms a crystalline solid at 25° C. and a halo-substituted diethyltoluenediamine.

### DETAILED DESCRIPTION OF INVENTION AND PREFERRED EMBODIMENTS

As used herein in reference to an organic compound, the term “aliphatic” means that the organic compound has a straight or branched chain structure and lacks any aryl or alicyclic ring moiety, wherein the chains comprise carbon atoms joined by respective single, double, or triple bonds and may optionally be interrupted by one or more heteroatoms, typically selected from oxygen, nitrogen, and sulfur heteroatoms, and the carbon atom members of the chains may each optionally be substituted with one or more organic groups that lack any aryl or alicyclic ring moiety, typically selected from alkyl, alkoxyl, hydroxyalkyl, cycloalkyl, alkoxyalkyl, haloalkyl.

As used herein in reference to an organic compound, the term “alicyclic” means that the compound comprises one or more non-aromatic ring moieties and lacks any aryl ring moiety, wherein the members of the one or more non-aromatic ring moieties comprise carbon atoms, each of the one or more non-aromatic ring moieties may optionally be interrupted by one or more heteroatoms, typically selected from oxygen, nitrogen, and sulfur heteroatoms, and the carbon atom members of the one or more non-aromatic ring moieties may each optionally be substituted with one or more non-aryl organic groups, typically selected from alkyl, alkoxyl, hydroxyalkyl, cycloalkyl, alkoxyalkyl, haloalkyl.

As used herein, the term “alkoxy” means a saturated straight or branched alkyl ether radical, more typically a (C₁-C₂₂) alkyl ether radical, such as, for example, methoxy, ethoxy, n-propoxy, isopropoxy, n-butoxy, tert-butoxy, sec-butoxy, n-pentoxy, and nonoxy.

As used herein, the term “alkoxyalkyl” means an alkyl radical that is substituted with one or more alkoxy substituents, more typically a (C₁-C₂₂) alkyloxy (C₁-C₆) alkyl radical, such as methoxymethyl, and ethoxybutyl.

As used herein, the term “alkyl” means a monovalent straight or branched saturated hydrocarbon radical, more typically, a monovalent straight or branched saturated (C₁-C₂₂) hydrocarbon radical, such as, for example, methyl, ethyl, n-propyl, isopropyl, n-butyl, isobutyl, tert-butyl, n-hexyl, n-octyl, and n-hexadecyl.

As used herein, the terminology “amine hydrogen equivalent weight” in reference to an amine compound bearing given amino functional group(s) means the quantity determined by dividing the mass of the amine compound by the product of the molecular weight of the amine compound and the reactivity index of the amino functional groups of the amine compound, where the “reactivity index” of the amine functional groups of the amine compound is the maximum number of bonds that the amino functional groups of the amine compound can theoretically form. For example, an amine compound bearing two primary amine groups per molecule of the amine compound has a reactivity index of 4, that is, the product of 2 amino functional groups per molecule of the amine compound multiplied times 2 active hydrogens per amino functional group.

As used herein in reference to an organic compound, the term “aromatic” means that the organic compound that comprises one or more one aryl moieties, which may each optionally be interrupted by one or more heteroatoms, typically selected from oxygen, nitrogen, and sulfur heteroatoms, and one or more of the carbon atoms of one or more one aryl moieties may optionally be substituted with one or more organic groups, typically selected from alkyl, alkoxyl, hydroxyalkyl, cycloalkyl, alkoxyalkyl, haloalkyl, aryl, alkaryl, aralkyl.

As used herein, the term “aryl” means cyclic, coplanar 5- or 6-membered organic group having a delocalized, conjugated TT system, with a number of TT electrons that is equal to 4n+2, where n is 0 or a positive integer, including compounds where each of the ring members is a carbon atom, such as benzene, compounds where one or more of the ring members is a heteroatom, typically selected from oxygen, nitrogen and sulfur atoms, such as furan, pyridine, imidazole, and thiophene, and fused ring systems, such as naphthalene, anthracene, and fluorene, wherein one or more of the ring carbons may be substituted with one or more organic groups, typically selected from alkyl, alkoxyl, hydroxyalkyl, cycloalkyl, alkoxyalkyl, haloalkyl, aryl, alkaryl, halo groups, such as, for example, phenyl, methylphenyl, trimethylphenyl, nonylphenyl, chlorophenyl, or trichloromethylphenyl.

As used herein, the terminology “(Cₙ-Cₘ)” in reference to an organic group, wherein n and m are each integers, indicates that the group may contain from n carbon atoms to m carbon atoms per group.

The terms “cure” and “curing” as used herein may include polymerizing and/or cross-linking of the curable resin composition.

As used herein, the term “curing agent” means a compound or complex that is capable of dissociating to provide one or more species capable of initiating polymerization of the curable resin component of the curable resin composition of the present invention.

As used herein, the term “cycloalkyl” means a saturated (C₅-C₂₂) hydrocarbon radical that includes one or more cyclic alkyl rings, such as, for example, cyclopentyl, cyclooctyl, and adamantanyl.

As used herein, “epoxide group” means a vicinal epoxy group, i.e., a 1,2-epoxy group.

As used herein, the term “fiber” has its ordinary meaning as known to those skilled in the art and may include one or more fibrous materials adapted for the reinforcement of composites, which may take the form of any of particles, flakes, whiskers, short fibers, continuous fibers, sheets, plies, and combinations thereof.

As used herein, the terminology “fiber pre-form” means assembly of fibers, layers of fibers, fabric or layers of fabric plies configured to receive a liquid curable resin composition in a resin infusion process.

As used herein, the term “halo” means a halogen radical, i.e., a chloro, fluoro, bromo, or iodo group.

As used herein, the term “haloalkyl” means an alkyl radical that is substituted with one or more halo substituents, such as chloromethyl, trichloromethyl, and trifluoromethyl.

As used herein, the term “hydroxyalkyl” means an alkyl radical, more typically a (C₁-C₂₂) alkyl radical, that is substituted with one or more hydroxyl groups, such as for example, hydroxymethyl, hydroxyethyl, hydroxypropyl, and hydroxydecyl.

As referred to herein, a “non-crimp fabric” or “NCF” means a fabric comprising of two or more plies of unidirectional fibers, the fibers of which may be stitched, knitted, braided, discontinuous fibers, or an adhesively bonded chopped fiber mat.

As used herein, the term “prepreg” means a fiber reinforcement that has been pre-impregnated, fully or partially, with curable resin composition or a fabric made from woven tows of fibers that have been pre-impregnated with resin curable resin composition.

Compounds suitable as the at least one multifunctional aromatic amine of the curing agent composition of the present invention and/or of the curable epoxy resin composition of the present invention are those that comprise at least one aromatic moiety per molecule and at least two amino groups per molecule, wherein each of the amino groups is borne by a respective carbon atom of the one or more aromatic moieties, and may, optionally, be further substituted on one or more carbon atoms of one or more aromatic moieties with one or more steric blocking groups and/or one or more electron withdrawing groups and that, when in pure form, form a crystalline solid at 25° C.

In one embodiment, the at least one multifunctional aromatic amine comprises a compound according to structure (I):

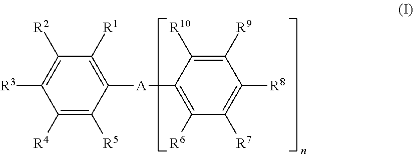

wherein:

A is amino or a divalent linking group, and

n is 0 or 1, wherein:
if A is amino, then:
   n=0,
   R¹ and R⁵ are each H, a steric blocking group or an electron withdrawing group, and
   R², R³, and R⁴, are each H, a steric blocking group, an electron withdrawing group, or amino, provided that at least one of R², R³, and R⁴ is amino, and
if A is a divalent linking group, then:
n=1,
R¹, R⁵, R¹⁰, and R⁶ are each H or a steric blocking group, wherein R¹ and R¹⁰ or R⁵ and R⁶ may be replaced by a covalent bond between the respective carbon atoms of the respective aromatic rings,
R², R³, R⁴, R⁷, R⁸, and R⁹ are each H, a steric blocking group, an electron withdrawing group, or amino, provided that at least one of R², R³, and R⁴ is amino, and if R² or R⁴ is amino, then R⁷ or R⁹ is amino, and if R³ is amino, then R⁸ is amino.

Suitable divalent linking groups include alkylene, sulphone, aryldioxy, and fluorenenyl radicals.

Suitable steric hindering groups include alkyl groups, more typically (C₁-C₆)alkyl groups, even more typically methyl, ethyl, propyl, isopropyl, or isobutyl.

Suitable electron withdrawing groups include halo, haloalkyl, —SO₃⁻, —NO₂, —CHO,

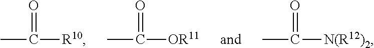

wherein:
R¹⁰ is alkyl, more typically (C₁-C₆)alkyl,
R¹¹ is alkyl, more typically (C₁-C₆)alkyl, and
R¹² is H, alkyl, more typically (C₁-C₆)alkyl.

In one embodiment, the multifunctional aromatic amine comprises a compound according to structure (I) wherein A is an amino group and n is 0, such as a diaminobenzene. Suitable diamionobenzenes include, for example, 1,3-diaminobenzene, and 4-methyl-1,3-diaminobenzene.

In one embodiment, the multifunctional aromatic amine comprises a compound according to structure (I) wherein A is a divalent aryldioxy linking group such, as a bis-aminophenoxy benzene. Suitable bis-aminophenoxy benzenes include, for example, 1,3-bis(3-aminophenoxy)benzene and 1,4-bis(4-aminophenoxy)-2-phenylbenzene.

In one embodiment, the multifunctional aromatic amine comprises a compound according to structure (I) wherein A is a divalent alkylene linking group, such as an alkylene bis-aniline. Suitable alkylene bis-anilines include, for example:

4,4′-methylene-bis-(2,6-diethylaniline) (“MDEA”),

4,4′-Methylene-bis-(3-chloro-2,6-diethylaniline (“M-CDEA”),

4,4′-Methylene-bis-(2-isopropyl-6-methylaniline) (“M-MIPA”), and

4,4 Methylenebis(2,6-diisopropylaniline) (“M-DIPA”).

In one embodiment, the multifunctional aromatic amine comprises a compound according to structure (I) wherein A is a divalent alkylene linking group and R¹ and R¹⁰ or R⁵ and R⁶ may be replaced by a covalent bond between the respective carbon atoms of the respective aromatic rings, such as a diaminofluorene. Suitable diaminofluorenes include, for example 2,7-diaminofluorene.

In one embodiment, the multifunctional aromatic amine comprises a compound according to structure (I) wherein A is divalent fluorenyl linking group, such as a bis-aminoaryl fluorene. Suitable bis-aminoaryl fluorenes include, for example:

9,9-bis-(4-aminophenyl)-fluorene (“AF”),

9,9-bis-(3-methyl-4-aminophenyl)-fluorene (“BMAF”)) and

9,9-Bis(4-amino-3-chlorophenyl)-fluorene (“CAF”).

In one embodiment, the multifunctional aromatic amine comprises a compound according to structure (I) wherein A is divalent sulphone linking group, such as a di(aminoaryl)sulphone. Suitable di(aminoaryl)sulphones include, for example, 3,3-diaminodiohenylsulphone (“3,3-DDS”), and 4,4-diaminodiohenylsulphone (“4,4-DDS”).

In one embodiment, the curing agent comprises one or more multifunctional aromatic amine selected from the group consisting of MDEA, M-CDEA, MDEA, M-MIPA, AF, BMAF, CAF, 3,3-DDS, and 4,4-DDS.

In one embodiment, the curing agent comprises one or more multifunctional aromatic amine selected from the group consisting of MDEA, M-CDEA, MDEA, CAF, and 3,3-DDS.

In one embodiment, the curing agent comprises one or more multifunctional aromatic amine selected from the group consisting of MDEA, M-CDEA, MDEA, and CAF.

Suitable multifunctional aromatic amines are known compounds and are commercially available from a number of sources.

The at least one halo-substituted diethyltoluenediamine comprises at least one compound according to formula (II):

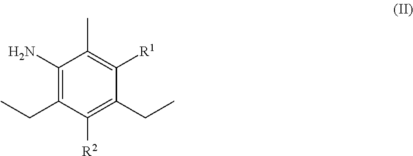

wherein:

R¹ is an amino group and R² is halo, or

R¹ is halo and R² is an amino group, or

a mixture thereof.

In one embodiment, the at least one halo-substituted diethyltoluenediamine is a compound according to formula (II), wherein R¹ is an amino group and R² is chloro, or R¹ is chloro and R² is an amino group, or a mixture thereof.

In one embodiment, the halo-substituted diethyltoluenediamine is selected from the group consisting of 6-chloro-3,5-diethyltoluene-2,4-diamine, 4-chloro-3,5-diethyltoluene-2,6-diamine, 6-chloro-3,5-diethyltoluene-2,4-diamine-4-chloro-3,5-diethyltoluene-2,6-diamine, and mixtures thereof.

In one embodiment, the amount of the at least one halo-substituted diethyltoluenediamine component of the curing agent composition of the present invention is effective to inhibit crystallization of the at least one multifunctional aromatic amine component of the curing agent composition of the present invention to the extent that the curing agent composition of the present invention remains liquid when maintained at temperature greater than or equal to 25° C. for greater than or equal to 5 days, more typically, for a time period of greater than or equal to 10 days, even more typically, for greater than or equal to 25 days.

In one embodiment, the curing agent composition of the present invention comprises, based on 100 pbw of the combined amount of the at least one multifunctional aromatic amine and at least one halo-substituted diethyltoluenediamine, from 10 to 90 pbw, more typically from 20 to 80 pbw, and even more typically from 40 to 60 pbw of the at least one multifunctional aromatic amine and from 10 to 90 pbw, more typically from 20 to 80 pbw, and even more typically from 40 to 60 pbw of the at least one halo-substituted diethyltoluenediamine.

In general, epoxy compounds suitable for use as the at least one epoxy compound component of the curable resin composition of the present invention are saturated or unsaturated aliphatic, cycloaliphatic, aromatic or heterocyclic compounds that have at least two epoxide group per molecule and include aromatic epoxy compounds, epoxy compounds, alicyclic epoxy compounds, and epoxy compounds.

Suitable aromatic epoxy compounds include aromatic compounds having two or more epoxide groups per molecule, including known compounds such as, for example: polyglycidal ethers of phenols and of polyphenols, such as diglycidyl resorcinol, 1,2,2-tetrakis(glycidyloxyphenyl) ethane, or 1,1,1-tris(glycidyloxyphenyl)methane, the diglycidal ethers of bisphenol A (bis(4-hydroxyphenyl)-2,2-propane), bisphenol F (bis(4-hydroxyphenyl)methane), bisphenol C (bis(4-hydroxyphenyl)-2,2-dichloroethylene), and bisphenol S (4,4′-sulfonyldiphenol), including oligomers thereof, fluorene ring-bearing epoxy compounds, naphthalene ring-bearing epoxy compounds, dicyclopentadiene-modified phenolic epoxy compounds, epoxidized novolac compounds, and epoxidized cresol novolac compounds, polyglycidal adducts of amines, such as N,N-diglycidal aniline, N,N,N′,N′-tetraglycidyl diaminodiphenylmethane (TGDDM), triglycidyl aminophenols (TGAP), triglycidyl aminocresol, or tetraglycidyl xylenediamine, or amino alcohols, such as triglycidal aminophenol, polyglycidal adducts of polycarboxylic acids, such as diglycidal phthalate, polyglycidal cyanurates, such as triglycidal cyanurate, copolymers of glycidal(meth)acrylates with copolymerizable vinyl compounds, such as styrene glycidal methacrylate.

Suitable epoxy compounds having two or more epoxide groups per molecule include known, commercially available compounds, such as N,N,N′,N′-tetraglycidyl diamino diphenylmethane (such as MY 9663, MY 720, and MY 721 from Huntsman), N,N,N′,N′-tetraglycidyl-bis(4-aminophenyl)-1,4-diiso-propylbenzene (such as EPON 1071 from Momentive); N,N,N′,N′-tetraclycidyl-bis(4-amino-3,5-dimethylphenyl)-1,4-diisopropylbenzene, (such as EPON 1072 from Momentive); triglycidyl ethers of p-aminophenol (such as MY 0510 from Hunstman); triglycidyl ethers of m-aminophenol (such as MY 0610 from Hunstman); diglycidyl ethers of bisphenol A based materials such as 2,2-bis(4,4′-dihydroxy phenyl) propane (such as DER 661 from Dow, or EPON 828 from Momentive, and Novolac resins preferably of viscosity 8-20 Pa s at 25° C.; glycidyl ethers of phenol Novolac resins (such as. DEN 431 or DEN 438 from Dow); di-cyclopentadiene-based phenolic novolac (such as Tactix® 556 from Huntsman); diglycidyl 1,2-phthalate; diglycidyl derivative of dihydroxy diphenyl methane (such as PY 306 from Huntsman).

Suitable alicyclic epoxy compounds having two or more epoxide groups per molecule, including known compounds such as, for example, bis(2,3-epoxy-cyclopentyl)ether, copolymers of bis(2,3-epoxy-cyclopentyl)ether with ethylene glycols, dicyclopentadiene diepoxide, 4-vinylcyclohexenedioxide, 3,4-epoxycyclohexylmethyl, 3,4-epoxycyclohexane carboxylate, 1,2,8,9-diepoxy limonene (limonene dioxide), 3,4-epoxy-6-methyl-cyclohexylmethyl, 3,4-epoxy-6-methylcyclohexane carboxylate, bis(3,4-epoxy-6-methylcyclohexylmethyl)adipate, 2-(7-oxabicyclo[4.1.0]hept-3-yl)spiro[1,3-dioxane-5,3′-[7]oxabicyclo[4.1.0]heptane], diepoxides of allyl cyclopentenyl ether, 1,4-cyclohexadiene diepoxide, 1,4-cyclohexanemethanol diglydical ether, bis(3,4-epoxycyclohexylmethyl) adipate, 3,4-epoxy-6-methylcyclohexane carboxylate, diglycidal 1,2-cyclohexane carboxylate, 3,4-epoxycyclohexylmethyl methacrylate, 3-(oxiran-2-yl)-7-oxabicyclo[4.1.0]heptane, bis(2,3-epoxypropyl) cyclohex-4-ene-1,2-dicarboxylate, 4,5-epoxytetrahydrophthalic acid diglycidyl ester, poly[oxy(oxiranyl-1,2-cyclohexanediyl)] α-hydro-ω-hydroxy-ether, bi-7-oxabicyclo[4.1.0]heptane. Suitable aliphatic epoxy compounds having two or more epoxide groups per molecule, including known compounds such as, for example: butanediol diglycidyl ether, epoxidized polybutadiene, dipentene dioxide, trimethylolpropane triglycidyl ether, bis[2-(2-butoxyethyoxy)ethyl)ethyl] adipate, hexanediol diglycidal ether, and hydrogenated bisphenol A epoxy resin. Suitable alicyclic epoxy compounds and aliphatic epoxy compounds include known, commercially available compounds, such as, for example: 3′,4′-epoxycyclohexanemethyl-3,4-epoxycyclohexylcarboxylate (CELLOXIDE™ 2021P resin (Daicel Corporation) and ARADITE CY 179 (Huntsman Advanced Materials)), bi-7-oxabicyclo[4.1.0]heptane (CELLOXIDE™ 8010 (Daicel Corporation)) 3:1 mixture of poly[oxy(oxiranyl-1,2-cyclohexanediyl)], α-hydro-ω-hydroxy-ether with 2-ethyl-2-(hydroxymethyl)-1,3-propanediol (EHPE 3150 (Daicel)).

The composition may optionally further comprise one or more monoepoxide compounds having one epoxide group per molecule, selected from aromatic monoepoxy compounds, monoalicyclic epoxy compounds, and aliphatic monoepoxy compounds. Suitable monoepoxide compounds, including known compounds such as, for example: saturated alicylic monoepoxides, such as 3,3′-bis(chloromethyl)oxacyclobutane, isobutylene oxide, styrene oxide, olefinic monoepoxides, such as cyclododecadiene monoepoxide, 3,4-epoxy-1-butene.

In one embodiment, the at least one epoxy compound of the curable resin composition of the present invention comprises one or more epoxy compounds selected from the diglycidal ether of bisphenol A and oligomers thereof, bisphenol F and oligomers thereof, tetraglycidyl diamino diphenyl methane, tri-glycidyl aminophenols, cresol novolac epoxy resins, and phenol novolac epoxy resins.

In one embodiment, the at least one epoxy compound of the curable resin composition of the present invention comprises one of, or a mixture of P-(2,3-epoxypropoxy)-N,N-bis(2, 3-epoxypropyl)aniline, M-(2,3-epoxypropoxy)-N,N-bis(2, 3-epoxypropyl)aniline, 4,4′-Methylenebis[N,N-bis(2,3-epoxypropyl)aniline] or Bis[4-(glycidyloxy)phenyl]methane.

In one embodiment, the curable resin composition of the present invention comprises, based on 100 parts by weight (“pbw”) of the combined amount of the at least one epoxy compound, at least one multifunctional aromatic amine, and at least one halo-substituted diethyltoluenediamine of the resin composition, from 10 to 90 pbw, more typically from 20 to 80 pbw, and even more typically from 30 to 70 pbw, of the one or more epoxy compounds.

In one embodiment, the curable resin composition of the present invention comprises, based on epoxy equivalents of the resin composition, from 0.25 to 0.09 amine hydrogen equivalent weight, more typically from 0.2 to 0.09 amine hydrogen equivalent weight, and even more typically from 0.15 to 0.1 amine hydrogen equivalent weight, of multifunctional aromatic amine per epoxy equivalent weight.

In one embodiment, the curable resin composition of the present invention comprises, based on 100 pbw of the combined amount of the at least one epoxy compounds, at least one multifunctional aromatic amine, and at least one halo-substituted diethyltoluenediamine of the resin composition, from 5 to 95 pbw, more typically from 15 to 85 pbw, and even more typically from 30 to 70 pbw, of the at least one multifunctional aromatic amine.

In one embodiment, the amount of the at least one halo-substituted diethyltoluenediamine component of the curable resin composition of the present invention is effective to inhibit crystallization of the at least one multifunctional aromatic amine component of the curable resin composition of the present invention to the extent that the curable resin composition of the present invention remains a single liquid phase, that is, without formation of crystals of the at least one multifunctional aromatic amine, when maintained at temperature greater than or equal to 25° C. for greater than or equal to 5 days, more typically, for greater than or equal to 10 days, even more typically, for greater than or equal to 20 days.

In one embodiment, the curable resin composition of the present invention comprises, based on 100 pbw of the combined amount of the at least one multifunctional aromatic amine curing agent and the at least one halo-substituted diethyltoluenediamine components of the curable resin composition, from 5 to 95 pbw, more typically from 15 to 85 pbw, and even more typically from 20 to 70 pbw, of the at least one halo-substituted diethyltoluenediamine.

The curable resin composition of the present invention may further comprise one or more additives in order to impart certain properties to the uncured composition or to the cured composite structure. The additives may be added to influence one or more of mechanical, rheological, electrical, optical, chemical, flame resistance and/or thermal properties of the cured or curable resin composition. Examples of such additives may include, but are not limited to, flame retardants, ultraviolet (UV) stabilizers, inorganic fillers, conductive particles or flakes, flow modifiers, thermal enhancers, density modifiers, toughening additives (such as core-shell particles, thermoplastic polymers), short fibers (inorganic or organic) and other optional additives commonly employed as additives for curable epoxy resin compositions, as is known in the art, and mixtures thereof.

In one embodiment, the curable resin composition of the present invention optionally further comprises, based on 100 pbw of the combined amount of the one or more epoxy compounds, at least one multifunctional aromatic amine, at least one halo-substituted diethyltoluenediamines, and additives of the resin composition, up to 50 pbw, more typically from 1 to 30 pbw, and even more typically from 1 to 10 pbw, of additives.

In one embodiment, curable resin composition of the present invention exhibits a viscosity of from 1-100000 centiPoise, more typically of from 1,000 to 100,000 Poise, as measured at 40° C. under steady state conditions using a Brookfield viscometer according to ASTM D2196.

In one embodiment, the viscosity of the curable resin composition of the present invention is from 15 to 50 centiPoise, more typically from 20 to 50 centiPoise, as measured at a temperature of 100° C. by parallel plate rheology according to ASTM D4440.

The curable resin composition of the present invention is useful as the matrix for a curable fiber reinforced resin matrix composite material, that is, a material, comprising fibers impregnated with the curable resin composition, from which to make cured fiber reinforced resin matrix articles.

Fibers suitable for use as the fiber component of the curable fiber reinforced resin matrix composite material include, for example, carbon fibers, graphite fibers, glass fibers, such as E glass fibers, ceramic fibers, such as silicon carbide fibers, synthetic polymer fibers, such as aromatic polyamide fibers, polyimide fibers and polybenzoxazole fibers. The weight of a single layer or cross section of such fibers can vary from 50 to 600 g/m². In one embodiment, the fibers comprise carbon fibers, glass fibers, or both carbon fibers and glass fibers. In one embodiment, the fibers comprise carbon fibers, including, for example, carbon fibers that exhibit a tensile strength of greater than or equal to 3.5 GigaPascals (“GPa”) and a tensile modulus of greater than or equal to 200 GPa. Suitable carbon fibers are known materials that are commercially available, such as for, example, Tenax E STS40 F13 24 k fibers (Toho Tenax Co., Ltd.), and Torayca T800S fibers (Toray).

The curable fiber reinforced resin matrix composite material of the present invention typically comprises, based on 100 pbw of the curable fiber reinforced resin matrix composite material, from 50 to 80 pbw, and more typically, from 60 to 70 pbw, of fibers, and from 20 to 50 pbw, more typically 30 to 40 pbw of the curable resin composition of the present invention.

In the present invention, the epoxy resin, multifunctional aromatic amine, and halo-substituted diethyltoluenediamine are mixed together before associating the curable resin composition with the fibers. The resin may be preheated to adjust the unmixed and mixed viscosities as appropriate.

Selection of the at least one epoxy compound, the at least one multifunctional aromatic amine, and the mix ratio of the epoxy compound, multifunctional aromatic amine, and halo-substituted diethyltoluenediamine allows flexibility to adjust of the properties of the curable epoxy resin and cured epoxy resin.

The structure of a suitable multifunctional amine cure agent is, to an extent, dictated by the reactivity of the formulation to be achieved and the choice of process by which the curable resin composition is introduced to the fiber reinforcement.

Special care needs to be given to maintaining a balance among the molecular structure of the curing agent and thus the reactivity of the curable resin composition, the temperature at which the resin is stored and maintained in a the resin pot that is in fluid communication with the mold, that is, the pot temperature, and the temperature at which the resin transfer process is to be conducted and the resin is to be cured, that is, the tool temperature.

A multifunctional aromatic amine curing agent in which the structure is selected to de-activate the amine functionality and thus increase the activation energy necessary for the curing reaction to take place are desirable where a “one part”, fully formulated curable epoxy compound is used in order to provide a manageable pot life for the composition.

Curable liquid resin compositions that contain epoxy compounds and multifunctional aromatic amine curing agents that offer good performance, including a good balance of reactivity and pot life, except for a tendency to phase separate into epoxy compound-rich and crystalline multifunctional aromatic amine phases during storage at ambient temperature. The crystalline amine phase must be dissolved and re-disbursed in the liquid resin formulation, for example, by mixing the resin formulation, with heating, prior to use. The need for such processing of such formulations immediately prior to use negates a significant part of the advantage of using a single component curable resin composition.

The curable resin composition of the present invention minimizes such process complexity and allows the balance among reactivity, toll temperature, and pot temperature to be more easily maintained.

The curing agent composition of the present invention and the curable resin composition of the present invention each exhibit resistance to recrystallization and precipitation of the multifunctional aromatic amine curing agent without requiring heating and re-mixing of the curable resin composition at elevated temperature immediately prior to use.

In one embodiment, the curable resin composition is combined with fiber reinforcement, for example, by introducing the resin composition into a mold that contains fiber reinforcement or by applying the resin composition to a fiber reinforcement in a filament winding process, and cured to form a fiber reinforced resin matrix composite article.

In one embodiment of the process of the present invention, the curing agent composition of the present invention and at least one epoxy compound are mixed to provide a curable resin composition according to the present invention, the curable resin composition is maintained in a reservoir at a pot temperature until the composition is combined with the fiber reinforcement.

In one embodiment of the process of the present invention, the curing agent composition of the present invention and at least one epoxy compound are maintain in separate reservoirs and mixed to provide a curable resin composition according to the present invention immediately prior to combining the resin composition with fiber reinforcement.

After combining the curable resin composition and the fiber reinforcement, the curable resin composition is cured to form a fiber reinforce resin matrix composite article.

In one embodiment, a composite material is formed by impregnating a fiber preform with the curable resin composition of the present application using a resin transfer molding process, such as a basic low (e.g., 10-20 Bars) pressure resin transfer molding (“RTM”) process, a vacuum assisted resin transfer molding (“VARTM”) process, or a high pressure (e.g., up to 150 Bars) resin transfer molding (“HP-RTM”) process, to form a fiber reinforced resin matrix composite material and subsequently cured to form a cured fiber reinforced resin matrix composite article.

The fiber pre-form may comprise, any desired configuration of reinforcing fibers, such as, for example, continuous fibers: plies of a unidirectional continuous fiber tape, a 3-dimensional woven fabric, a non-woven discontinuous fiber mat, or a non-crimp fabric.

In one embodiment the resin infusion process is an RTM process, or high pressure resin transfer molding (HP-RTM) process. RTM and HP-RTM are processes in which the curable resin composition is introduced into a closed mold which contains a dry fiber pre-form. The curable resin composition is injected into the mold that contains the fiber preform, which is maintained under low pressure or under vacuum. It is desirable to use a resin composition that exhibits a relatively low viscosity at the injection temperature, such as, for example, in the case of RTM, a viscosity of less than or equal to 5 Poise, more typically less than or equal to 1 Poise, at the injection temperature of 50 to 160° C., more typically 80 to130° C., in order to obtain the optimum mold filling and wetting of the fiber pre-form. Further, the resin system must maintain this low viscosity for a period of time sufficient to completely fill the mold and infuse the fiber preform. For RTM processing, such time is frequently measured in terms of the pot life of the resin, which can be defined as the time required for the resin to reach 5 Poise at a given temperature.

The fibrous preform is placed in a closed mold, which is typically heated to an initial tool temperature, such as from 20° C. to 220° C., followed by injection of the liquid curable resin composition into the mold to affect infusion of the liquid resin into the preform. The mold may be maintained at a selected dwell temperature such as from 20° C. to 220° C., during the infusion of curable resin composition into the fiber preform. After resin infusion is completed, the temperature of the mold is raised to affect curing of the curable resin composition component of the resin-infused preform, thereby forming a molded composite article. The resulting molded composite article can then be removed from the mold and post-cured as necessary.

Typical RTM processes operate at a pressure of, for example, 10-20 Bars injection pressure and pressure in the mold, pressure with a cycle time of 30 to 60 minutes.

In one embodiment, a composite material is formed by impregnating a fiber preform with the curable resin composition of the present application using a VARTM process. VARTM is a variation on the basic RTM process which operates in a manner that is generally similar to RTM, but in which a fiber preform is placed in a one-sided mold that is enclosed in a flexible vacuum bag.

A vacuum is applied while transferring liquid resin into the mold to force the liquid resin into the preform.

In one embodiment, a composite material is formed by impregnating a fiber preform with the curable resin composition of the present application using an HP-RTM process. HP-RTM is a variation on the basic RTM process that operates in a manner generally similar to RTM, but at higher pressure, such as, for example, 30-120 Bars injection pressure and pressure in the mold, with a shorter cycle time, such as, for example, less than 10 minutes.

In one embodiment, a composite material is formed by impregnating a fiber preform with the curable resin composition of the present application using a filament winding process.

In a filament winding process, the curable resin composition of the present invention is applied directly to the reinforcing fibers of a composite material as the fibers are wound around a mandrel using a winding head that is moved back and forth along the mandrel while rotating the mandrel. This technique allows a variety of fiber orientations, and consequently, a wide variety of interlocking angles of fibers, to be constructed.

The filament wound structure can be cured on the mandrel to form a hollow composite structure or can be removed from the mandrel prior to curing and flattened to form resin-impregnated fiber reinforced “blank” that used as a substrate in a way similar to resin-impregnated fiber reinforced pre-preg materials, such as by stacking layers of material in a selected orientation with respect to one another to form a “layup”, which is subsequently cured.

A filament wound fiber reinforced resin matrix composite blank according to the present invention can be “B-staged” by allowing the resin composition to react at ambient temperature for 4 to 8 hours, during which time the resin composition will reach a viscosity, typically about 50,000 to 300,000 Poise, to minimize flow of the resin composition from the material and allow the filament wound material to be more easily handled.

The fiber reinforced resin matrix composite material of the present invention is molded and cured to form a cured fiber reinforced resin matrix article, such as, for example, parts for aerospace, automotive, oil and gas field, wind turbine blade, and sporting goods applications.

In one embodiment, curable resin composition of the present invention has a pot life of from 1 to 9 hrs, more typically from 1 to 4 hrs, at a temperature of from 70 to 80° C., and is capable of being cured at a temperature of greater than or equal to 120° C., more typically of from 150° C. to 180° C., and even more typically of from 170° C. to 180° C., for a time period of less than or equal to 240 minutes, more typically of from 120 to 180 minutes, and even more typically of from 60 minutes to 120 minutes to provide a cured resin matrix.

### EXAMPLES

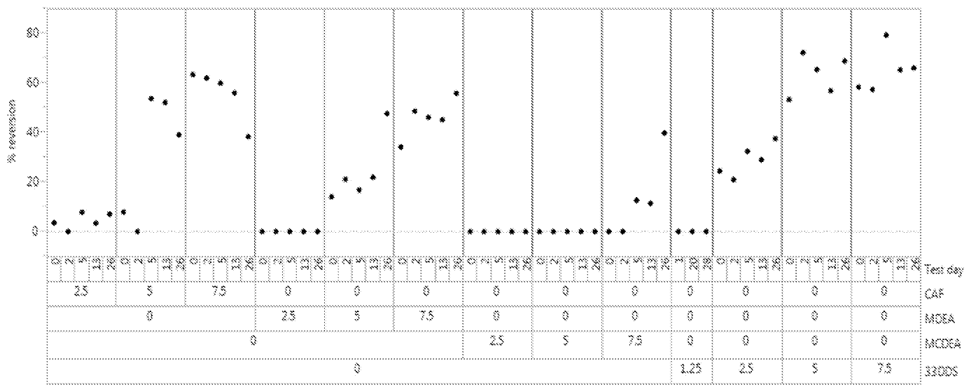

The compositions of EX1-EX18 were made by dissolving a multifunctional aromatic amine, either CAF, MDEA, MCDEA, or 3,3-DDS, in DETDA-CI at 120° C. for 20 mins. The amount of each multifunctional aromatic amine and DETDA-CI in each of the compositions of EX1-EX18 is given in TABLES 1-5 and in FIG. 1, each as pbw per 10 pbw of the composition.

The evolution of melt energy of the dissolved curing agent for each of the compositions was monitored over time using Differential Scanning Calorimetry, running on a −50 to 350° C. scan (+/−1° C./min modulation), and the % recrystallization of the dissolved curing agent was determined over time by normalising the melt energy observed over time for the samples with the melt energy of the pure multifunctional aromatic amine and the wt % in the mixture of the multifunctional aromatic amine.

Results are shown in in TABLES 1-5, as Melt Onset (° C.), as Melt Peak (° C.), Melt End Point (° C.), Melt Energy (J/g), Calculated Melt Energy (if fully reverted (J/g)), and % Reversion for each of the compositions of Examples 1-16, and in FIG. 1, in which the % Reversion values for each of the compositions of EX1-EX18 are plotted versus time.

The compositions of:

EX1 (1.25 pbw 3,3-DDS/8.75 pbw DETDA-C)I,

EX5 (2.5 pbw MCDEA/7.5 pbw DETDA-CI),

EX6 (5 pbw MCDEA/5 pbw DETDA-CI)

EX8 (2.5 pbw MDEA/7.5 pbw DETDA-CI), and

EX11 (2.5 pbw CAF/7.5 pbw DETDA-CI),

were each found to be resistant to recrystallization over the 26 day period investigated.

The compositions of:

EX2 (2.5 pbw 3,3-DDS 17.5 pbw DETDA-CI),

EX7 (7.5 pbw MCDEA 12.5 pbw DETDA-CI),

EX9 (5 pbw MDEA/5 pbw DETDA-CI), and

EX12 (5 pbw CAF/5 pbw DETDA-CI),

were each found to be resistant to recrystallization, but to a lesser extent and for a shorter period of time compared to the mixtures of EX1, EX5, EX6, EX8, and EX11.

TABLE 1
Melt Temperature, Melt Energy, and % Reversion, Day 0

| | | | | | | | | | | Calculated | |
|---|---|---|---|---|---|---|---|---|---|---|---|
| | | | | | | | | | | Melt | |
| | | | | | | | | Melt | | Energy | |
| | | | | | DETDA- | Melt | Melt | End | Melt | (fully | |
| | 33DDS | MCDEA | MDEA | CAF | Cl | Onset | Peak | Point | Energy | reverted | % |
| EX# | (pbw) | (pbw) | (pbw) | (pbw) | (pbw) | (° C.) | (° C.), | (° C.), | (J/g) | (J/g)) | Reversion |
| 1 | 1.25 | | | | 8.75 | — | — | — | — | 0 | 0 |
| 2 | 2.5 | — | — | — | 7.5 | 78.68 | 102.6 | 113.5 | 5.789 | 23.7025 | 24.42 |
| 3 | 5 | — | — | — | 5 | 94.3 | 127.32 | 143.22 | 25.25 | 47.405 | 53.26 |
| 4 | 7.5 | — | — | — | 2.5 | 112.47 | 138.6 | 152.92 | 41.46 | 71.1075 | 58.31 |
| 5 | — | 2.5 | — | — | 7.5 | — | — | — | 0 | 22.065 | 0 |
| 6 | — | 5 | — | — | 5 | — | — | — | 0 | 44.13 | 0 |
| 7 | — | 7.5 | — | — | 2.5 | — | — | — | 0 | 66.195 | 0 |
| 8 | — | — | 2.5 | — | 7.5 | — | — | — | 0 | 29.875 | 0 |
| 9 | — | — | 5 | — | 5 | 33.07 | 55.85 | 66.21 | 8.42 | 59.75 | 14.09 |
| 10 | — | — | 7.5 | — | 2.5 | 37.42 | 68.29 | 96.33 | 30.58 | 89.625 | 34.12 |
| 11 | — | — | — | 2.5 | 7.5 | 110.83 | 116.42 | 124.3 | 0.967 | 27.125 | 3.56 |
| 12 | — | — | — | 5 | 5 | 90.81 | 146.45 | 159.27 | 4.258 | 54.25 | 7.85 |
| 13 | — | — | — | 7.5 | 2.5 | 151.04 | 174.04 | 184.66 | 51.56 | 81.375 | 63.36 |
| 14 | 1 | — | — | — | — | 168.5 | 173.39 | 179.85 | 94.81 | 94.81 | 100 |
| 15 | — | 1 | — | — | — | 85.1 | 89.93 | 97.56 | 88.26 | 88.26 | 100 |
| 16 | — | — | 1 | — | — | 85.09 | 90 | 96.1 | 119.5 | 119.5 | 100 |
| 17 | — | — | — | 1 | — | 197.06 | 202.31 | 209.81 | 108.5 | 108.5 | 100 |
| 18 | 2.5 | — | — | — | 7.5 | | | | | 0 | |

TABLE 2
Melt Temperature, Melt Energy and % Reversion, Day 2

| | | | | | | | | | | Calculated | |
|---|---|---|---|---|---|---|---|---|---|---|---|
| | | | | | | | | | | Melt | |
| | | | | | | | | Melt | | Energy | |
| | | | | | DETDA- | Melt | Melt | End | Melt | (fully | |
| | 33DDS | MCDEA | MDEA | CAF | Cl | Onset | Peak | Point | Energy | reverted | % |
| EX# | (pbw) | (pbw) | (pbw) | (pbw) | (pbw) | (° C.) | (° C.) | (° C.) | (J/g) | (J/g)) | Reversion |
| 1 | 1.25 | | | | 8.75 | — | — | — | — | 0 | 0 |
| 2 | 2.5 | — | — | — | 7.5 | 76.88 | 101.47 | 119 | 4.958 | 23.7025 | 20.92 |
| 3 | 5 | — | — | — | 5 | 92.39 | 129.62 | 174 | 34.24 | 47.405 | 72.23 |
| 4 | 7.5 | — | — | — | 2.5 | 41 | 144.49 | 157.4 | 40.75 | 71.1075 | 57.31 |
| 5 | — | 2.5 | — | — | 7.5 | — | — | — | 0 | 22.065 | 0 |
| 6 | — | 5 | — | — | 5 | — | — | — | 0 | 44.13 | 0 |
| 7 | — | 7.5 | — | — | 2.5 | — | — | — | 0 | 66.195 | 0 |
| 8 | — | — | 2.5 | — | 7.5 | — | — | — | 0 | 29.875 | 0 |
| 9 | — | — | 5 | — | 5 | 31.28 | 52.76 | 64.95 | 12.58 | 59.75 | 21.05 |
| 10 | — | — | 7.5 | — | 2.5 | 48.21 | 69.66 | 78.83 | 43.52 | 89.625 | 48.56 |
| 11 | — | — | — | 2.5 | 7.5 | — | — | — | — | 27.125 | 0 |
| 12 | — | — | — | 5 | 5 | — | — | — | — | 54.25 | 0 |
| 13 | — | — | — | 7.5 | 2.5 | 148.83 | 175.91 | 183.9 | 50.45 | 81.375 | 62.00 |
| 14 | 1 | — | — | — | — | 168.5 | 173.39 | 179.9 | 94.81 | 94.81 | 100 |
| 15 | — | 1 | — | — | — | 85.1 | 89.93 | 97.56 | 88.26 | 88.26 | 100 |
| 16 | — | — | 1 | — | — | 85.09 | 90 | 96.1 | 119.5 | 119.5 | 100 |
| 17 | — | — | — | 1 | — | 197.06 | 202.31 | 209.8 | 108.5 | 108.5 | 100 |
| 18 | 2.5 | — | — | — | 7.5 | | | | | 0 | 100 |

TABLE 3
Melt Temperature, Melt Energy and % Reversion, Day 5

| | | | | | | | | | | Calculated | |
|---|---|---|---|---|---|---|---|---|---|---|---|
| | | | | | | | | | | Melt | |
| | | | | | | | | Melt | | Energy | |
| | | | | | DETDA- | Melt | Melt | End | Melt | (fully | |
| | 33DDS | MCDEA | MDEA | CAF | Cl | Onset | Peak | Point | Energy | reverted | % |
| EX# | (pbw) | (pbw) | (pbw) | (pbw) | (pbw) | (° C.) | (° C.) | (° C.) | (J/g) | (J/g)) | Reversion |
| 1 | 1.25 | | | | 8.75 | — | — | — | — | 0 | 0 |
| 2 | 2.5 | — | — | — | 7.5 | 68.5 | 99.99 | 175.2 | 7.6826 | 23.7025 | 32.417 |
| 3 | 5 | — | — | — | 5 | 101.58 | 135.32 | 151.53 | 31 | 47.405 | 65.39 |
| 4 | 7.5 | — | — | — | 2.5 | 125.72 | 151.3 | 160.4 | 56.36 | 71.1075 | 79.26 |
| 5 | — | 2.5 | — | — | 7.5 | | | | 0 | 22.065 | 0 |
| 6 | — | 5 | — | — | 5 | | | | 0 | 44.13 | 0 |
| 7 | — | 7.5 | — | — | 2.5 | 53.35 | 70.97 | 81.2 | 8.392 | 66.195 | 12.68 |
| 8 | — | — | 2.5 | — | 7.5 | | | | | 29.875 | 0 |
| 9 | — | — | 5 | — | 5 | 29 | 57 | 72 | 10 | 59.75 | 16.74 |
| 10 | — | — | 7.5 | — | 2.5 | 38.48 | 68.47 | 80.27 | 41.35 | 89.625 | 46.14 |
| 11 | — | — | — | 2.5 | 7.5 | 108 | 115 | 122 | 2.1 | 27.125 | 7.74 |
| 12 | — | — | — | 5 | 5 | 113.3 | 149.02 | 159.24 | 29.1 | 54.25 | 53.64 |
| 13 | — | — | — | 7.5 | 2.5 | 146.23 | 173.24 | 182.63 | 48.8 | 81.375 | 59.97 |
| 14 | 1 | — | — | — | — | 168.5 | 173.39 | 179.85 | 94.81 | 94.81 | 100 |
| 15 | — | 1 | — | — | — | 85.1 | 89.93 | 97.56 | 88.26 | 88.26 | 100 |
| 16 | — | — | 1 | — | — | 85.09 | 90 | 96.1 | 119.5 | 119.5 | 100 |
| 17 | — | — | — | 1 | — | 197.06 | 202.31 | 209.81 | 108.5 | 108.5 | 100 |
| 18 | 2.5 | — | — | — | 7.5 | | | | | 0 | 100 |

TABLE 4
Melt Temperature, Melt Energy and % Reversion, Day 13

| | | | | | | | | | | Calculated | |
|---|---|---|---|---|---|---|---|---|---|---|---|
| | | | | | | | | | | Melt | |
| | | | | | | | | Melt | | Energy | |
| | | | | | DETDA- | Melt | Melt | End | Melt | (fully | |
| | 33DDS | MCDEA | MDEA | CAF | Cl | Onset | Peak | Point | Energy | reverted | % |
| EX# | (pbw) | (pbw) | (pbw) | (pbw) | (pbw) | (° C.) | (° C.) | (° C.) | (J/g) | (J/g)) | Reversion |
| 1 | 1.25 | | | | 8.75 | — | — | — | — | 0 | 0 |
| 2 | 2.5 | — | — | — | 7.5 | 63.7 | 95.03 | 123.46 | 6.863 | 23.7025 | 28.95 |
| 3 | 5 | — | — | — | 5 | 92.87 | 130.83 | 152.01 | 26.93 | 47.405 | 56.81 |
| 4 | 7.5 | — | — | — | 2.5 | 125.64 | 148.16 | 175.73 | 46.37 | 71.1075 | 65.21 |
| 5 | — | 2.5 | — | — | 7.5 | — | — | — | 0 | 22.065 | 0 |
| 6 | — | 5 | — | — | 5 | — | — | — | 0 | 44.13 | 0 |
| 7 | — | 7.5 | — | — | 2.5 | 46.22 | 68.41 | 79.11 | 7.53 | 66.195 | 11.37 |
| 8 | — | — | 2.5 | — | 7.5 | | | | 0 | 29.875 | 0 |
| 9 | — | — | 5 | — | 5 | 33.75 | 58.6 | 75.51 | 13.1 | 59.75 | 21.92 |
| 10 | — | — | 7.5 | — | 2.5 | 42.5 | 68.05 | 79.78 | 40.46 | 89.625 | 45.14 |
| 11 | — | — | — | 2.5 | 7.5 | 111.08 | 115.85 | 123.92 | 0.91 | 27.125 | 3.35 |
| 12 | — | — | — | 5 | 5 | 109.8 | 150.12 | 161.95 | 28.24 | 54.25 | 52.06 |
| 13 | — | — | — | 7.5 | 2.5 | 149.62 | 175.59 | 184.47 | 45.46 | 81.375 | 55.86 |
| 14 | 1 | — | — | — | — | 168.5 | 173.39 | 179.85 | 94.81 | 94.81 | 100 |
| 15 | — | 1 | — | — | — | 85.1 | 89.93 | 97.56 | 88.26 | 88.26 | 100 |
| 16 | — | — | 1 | — | — | 85.09 | 90 | 96.1 | 119.5 | 119.5 | 100 |
| 17 | — | — | — | 1 | — | 197.06 | 202.31 | 209.81 | 108.5 | 108.5 | 100 |
| 18 | 2.5 | — | — | — | 7.5 | | | | | 0 | 100 |

TABLE 5
Melt Temperature, Melt Energy and % Reversion, Day 26

| | | | | | | | | | | Calculated | |
|---|---|---|---|---|---|---|---|---|---|---|---|
| | | | | | | | | | | Melt | |
| | | | | | | | | Melt | | Energy | |
| | | | | | DETDA- | Melt | Melt | End | Melt | (fully | |
| | 33DDS | MCDEA | MDEA | CAF | Cl | Onset | Peak | Point | Energy | reverted | % |
| EX# | (pbw) | (pbw) | (pbw) | (pbw) | (pbw) | (° C.) | (° C.) | (° C.) | (J/g) | (J/g)) | Reversion |
| 1 | 1.25 | | | | 8.75 | | | | | | |
| 2 | 2.5 | — | — | — | 7.5 | 72.59 | 107.69 | 126.58 | 8.883 | 23.7025 | 37.48 |
| 3 | 5 | — | — | — | 5 | 108.14 | 140.53 | 158.89 | 32.64 | 47.405 | 68.85 |
| 4 | 7.5 | — | — | — | 2.5 | 131.11 | 150.84 | 161.53 | 46.97 | 71.1075 | 66.05 |
| 5 | — | 2.5 | — | — | 7.5 | — | — | — | 0 | 22.065 | 0 |
| 6 | — | 5 | — | — | 5 | — | — | — | 0 | 44.13 | 0 |
| 7 | — | 7.5 | — | — | 2.5 | 37.71 | 59.71 | 77.45 | 26.37 | 66.195 | 39.84 |
| 8 | — | — | 2.5 | — | 7.5 | — | — | — | 0 | 29.875 | 0 |
| 9 | — | — | 5 | — | 5 | 30.37 | 55.77 | 75.24 | 28.44 | 59.75 | 47.60 |
| 10 | — | — | 7.5 | — | 2.5 | 46.8 | 68.41 | 78.22 | 49.97 | 89.625 | 55.75 |
| 11 | — | — | — | 2.5 | 7.5 | 105.71 | 111.7 | 121.29 | 1.917 | 27.125 | 7.067 |
| 12 | — | — | — | 5 | 5 | 101.75 | 138.63 | 151.25 | 21.17 | 54.25 | 39.02 |
| 13 | — | — | — | 7.5 | 2.5 | 145.82 | 169.67 | 178.9 | 31.21 | 81.375 | 38.35 |
| 14 | 1 | — | — | — | — | 168.5 | 173.39 | 179.85 | 94.81 | 94.81 | 100 |
| 15 | — | 1 | — | — | — | 85.1 | 89.93 | 97.56 | 88.26 | 88.26 | 100 |
| 16 | — | — | 1 | — | — | 85.09 | 90 | 96.1 | 119.5 | 119.5 | 100 |
| 17 | — | — | — | 1 | — | 197.06 | 202.31 | 209.81 | 108.5 | 108.5 | 100 |
| 18 | 2.5 | — | — | — | 7.5 | | | | | 0 | 100 |

1. A curing agent composition, comprising:
at least one multifunctional aromatic amine that forms a crystalline solid at 25° C., and
at least one halo-substituted diethyltoluenediamine, in an amount effective to inhibit crystallization of the at least one multifunctional aromatic amine.

2. The composition of claim 1, wherein the at least one multifunctional aromatic amine comprises a compound according to structure (I): 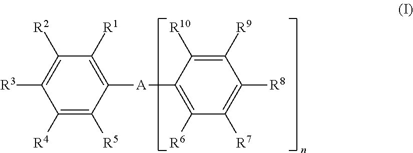
wherein:
A is amino or a divalent linking group, and
n is 0 or 1, wherein:
if A is amino, then:
n=0,
R¹ and R⁵ are each H, a steric blocking group or an electron withdrawing group, and
R², R³, and R⁴, are each H, a steric blocking group, an electron withdrawing group, or amino, provided that at least one of R², R³, and R⁴ is amino, and
if A is a divalent linking group, then:
n=1,
R¹, R⁵, R¹⁰, and R⁶ are each H or a steric blocking group, wherein R¹ and R¹⁰ or R⁵ and R⁶ may be replaced by a covalent bond between the respective carbon atoms of the respective aromatic rings,
R², R³, R⁴, R⁷, R⁸, and R⁹ are each H, a steric blocking group, an electron withdrawing group, or amino, provided that at least one of R², R³, and R⁴ is amino, and if R² or R⁴ is amino, then R⁷ or R⁹ is amino, and if R³ is amino, then R⁸ is amino.

3. The composition of claim 1, wherein the at least one multifunctional aromatic amine comprises one or more compounds selected from the group consisting of 4,4′-methylene-bis-(2,6-diethylaniline), 4,4′-methylene-bis-(3-chloro-2,6-diethylaniline, 9,9-bis(4-amino-3-chlorophenyl)-fluorene, 3,3-diaminodiohenylsulphone, and 4,4-diaminodiohenylsulphone.

4. The composition of claim 1, wherein at least one halo-substituted diethyltoluenediamine comprises a compound according to formula (II): 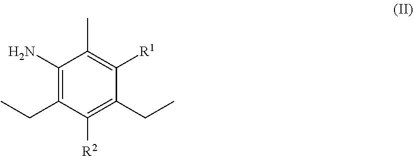
wherein:
R¹ is an amino group and R² is halo, or
R¹ is halo and R² is an amino group, or
a mixture thereof.

5. The composition of claim 1, wherein at least one halo-substituted diethyltoluenediamine is selected from the group consisting of 6-chloro-3,5-diethyltoluene-2,4-diamine, 4-chloro-3,5-diethyltoluene-2,6-diamine, 6-chloro-3,5-diethyltoluene-2,4-diamine-4-chloro-3,5-diethyltoluene-2,6-diamine, or a mixture thereof.

6. The composition of claim 1, wherein the amount effective to inhibit crystallization is, based on 100 pbw of the combined amount of the at least one multifunctional aromatic amine and at least one halo-substituted diethyltoluenediamine, from 10 to 90 pbw of the at least one multifunctional aromatic amine and from 10 to 90 pbw of the at least one halo-substituted diethyltoluenediamine.

7. A method for inhibiting phase separation of a curing agent composition, comprising adding to a curing agent composition that comprises at least one multifunctional aromatic amine that forms a crystalline solid at 25° C., at least one halo-substituted diethyltoluenediamine in an amount effective to inhibit crystallization of the at least one multifunctional aromatic amine.

8. A process for making a curable resin composition, comprising mixing the curing agent composition of claim 1 and at least one epoxy compound having at least two epoxide groups per molecule of the epoxy compound.

9. A curable resin composition, comprising:
at least one epoxy compound having at least two epoxide groups per molecule of the epoxy compound,
at least one multifunctional aromatic amine that is that forms a crystalline solid at 25° C., and
at least one halo-substituted diethyltoluenediamine, in an amount effective to inhibit crystallization of the at least one multifunctional aromatic amine.

10. A method for inhibiting phase separation of a curable resin composition, comprising adding, to a curable resin composition that comprises at least one epoxy compound having at least two epoxide groups per molecule of the epoxy compound and at least one multifunctional aromatic amine that is that is a crystalline solid at 25° C., at least one halo-substituted diethyltoluenediamine in an amount effective to inhibit crystallization of the at least one multifunctional aromatic amine.

11. A curable fiber reinforced resin matrix composite article, comprising: fibers and a curable resin composition according to claim 4.
